(19) United States
(12) Patent Application Publication
(10) Pub. No.: US 2026/0325087 A1
(43) Pub. Date: Oct. 22, 2026

(54) X-RAY CT APPARATUS, CONTROL METHOD OF HIGH-VOLTAGE GENERATION DEVICE, AND STORAGE MEDIUM

(71) Applicant: CANON MEDICAL SYSTEMS CORPORATION, Otawara-shi (JP)

(72) Inventor: Masaharu TSUYUKI, Nasushiobara (JP)

(73) Assignee: CANON MEDICAL SYSTEMS CORPORATION, Otawara-shi (JP)

(21) Appl. No.: 19/544,012

(22) Filed: Feb. 19, 2026

(30) Foreign Application Priority Data
Apr. 22, 2025 (JP) 2025-070315

(51) Int. Cl. A61B 6/40; A61B 6/03

(52) U.S. Cl. CPC A61B 6/405; A61B 6/032

(57) ABSTRACT

An X-ray computed tomography apparatus according to an embodiment includes an X-ray, a high-voltage generation unit, and processing circuitry. The X-ray tube includes an anode, a filament, and a grid electrode disposed between the anode and the filament. The high-voltage generation unit includes an inverter and is configured to apply a tube voltage between the anode and the filament. The processing circuitry is configured to enable an output voltage of the inverter when X-ray imaging is started and enable a grid voltage during a rise period of the tube voltage to inhibit a tube current, and is configured to, when the X-ray imaging is ended, disable the output voltage of the inverter and discharge electric charge remaining in the high-voltage generation unit.

### CROSS-REFERENCE TO RELATED APPLICATIONS

This application is based upon and claims the benefit of priority from Japanese Patent Application No. 2025-070315, filed Apr. 22, 2025, the entire contents of which are incorporated herein by reference.

### FIELD

Embodiments disclosed in this specification and the drawings relate to an X-ray computed tomography (CT) apparatus, a control method for a high-voltage generation device, and a storage medium.

### BACKGROUND

In an X-ray computed tomography (CT) apparatus, power is supplied from an X-ray high-voltage device to an X-ray tube. In the X-ray high-voltage device, an alternating-current (AC) voltage is subjected to AC/direct-current (DC) conversion, then converted to a high frequency by an inverter, and the voltage is raised through a transformer. The raised AC voltage is then supplied to the X-ray tube through rectifier circuitry.

In the X-ray high-voltage device, the output voltage of the inverter is enabled or disabled to enable or disable the emission of X-rays from the X-ray tube. Meanwhile, the rectifier circuitry has a capacitor. In such a configuration, charging and discharging of the capacitor takes time, so that the rise and fall of the tube voltage and tube current of the X-ray tube also take time. Therefore, during the time, X-rays that do not contribute to image formation are irradiated, resulting in exposure of the subject.

FIG. 8 is a diagram illustrating a circuitry configuration of an X-ray high-voltage device 14z according to the related art. FIG. 9 is a diagram illustrating temporal changes in the output voltage of an inverter INV and the tube voltage and tube current of an X-ray tube 11z according to the related art. In the X-ray tube 11z, X-ray radiation from the X-ray tube 11z can be enabled and disabled by enabling and disabling the output voltage of the inverter INV while a filament F is being heated. The tube voltage (kV) is the voltage between an anode A and the filament F. The tube current (mA) is the current flowing through the X-ray tube 11z.

When the output voltage of the inverter INV is enabled, the tube voltage and tube current do not rise immediately but take a predetermined time to rise. On the other hand, when the output voltage of the inverter INV is disabled, the tube voltage and tube current do not fall immediately but take a predetermined time to fall.

As illustrated in FIG. 9, a period M is a period in which the tube voltage and tube current have been sufficiently risen, and X-rays contributing to X-ray images are emitted. On the other hand, a period N is a period in which the tube voltage and tube current are rising and falling, and X-rays not contributing to X-ray images are emitted.

### BRIEF DESCRIPTION OF THE DRAWINGS

FIG. 1 is a block diagram illustrating a configuration example of an X-ray computed tomography (CT) apparatus according to a first embodiment;

FIG. 2 is a diagram illustrating a circuitry configuration of an X-ray high-voltage device and an X-ray tube according to the first embodiment;

FIG. 3 is a diagram illustrating temporal changes in the output voltage of an inverter and a tube current according to the first embodiment;

FIG. 4 is a diagram illustrating temporal changes in the output voltage of an inverter and a tube current according to a second embodiment;

FIG. 5 is a diagram illustrating temporal changes in the output voltage of an inverter and a tube current according to a third embodiment;

FIGS. 6A to 6C are diagrams illustrating the positional relationship between an X-ray tube and each member according to the third embodiment;

FIG. 7 is a diagram illustrating a circuitry configuration of an X-ray high-voltage device and an X-ray tube according to a fourth embodiment;

FIG. 8 is a diagram illustrating a circuitry configuration of an X-ray high-voltage device according to the related art; and

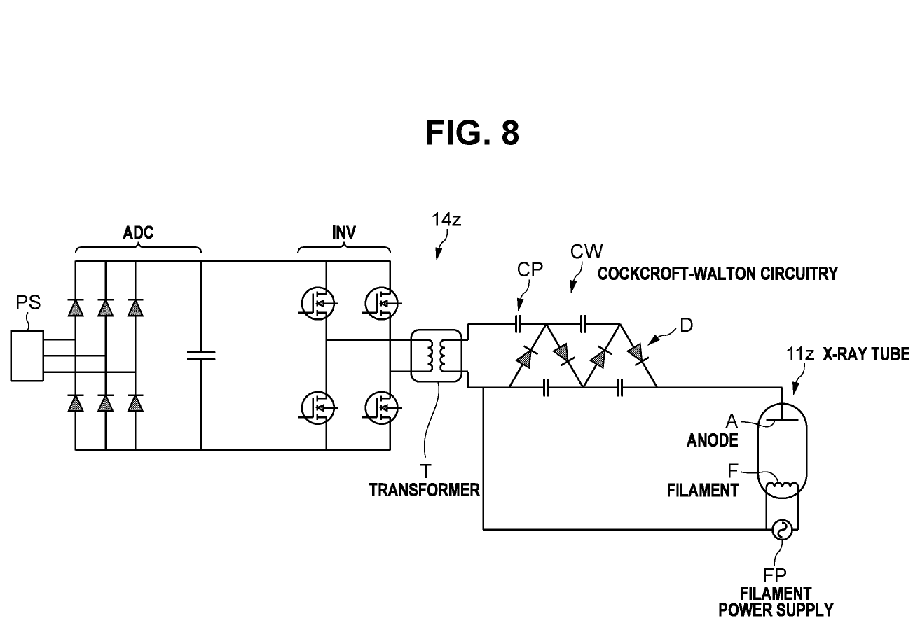

FIG. 9 is a diagram illustrating temporal changes in the output voltage of an inverter, and the tube voltage and tube current of an X-ray tube according to the related art.

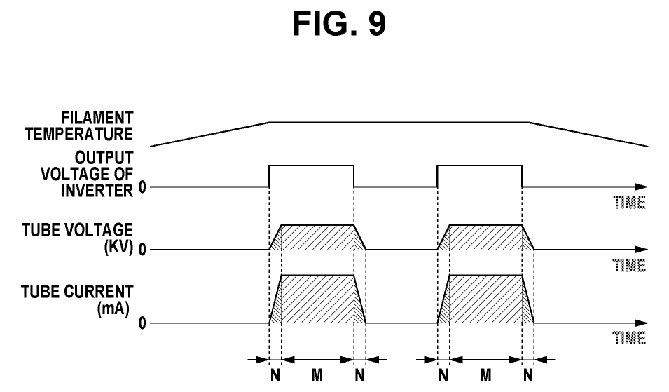

### DETAILED DESCRIPTION

An X-ray computed tomography apparatus according to an embodiment includes an X-ray, a high-voltage generation unit, and processing circuitry. The X-ray tube includes an anode, a filament, and a grid electrode disposed between the anode and the filament. The high-voltage generation unit includes an inverter and is configured to apply a tube voltage between the anode and the filament. The processing circuitry is configured to enable an output voltage of the inverter when X-ray imaging is started and enable a grid voltage during a rise period of the tube voltage to inhibit a tube current, and is configured to, when the X-ray imaging is ended, disable the output voltage of the inverter and discharge electric charge remaining in the high-voltage generation unit.

Various Embodiments will be described hereinafter with reference to the accompanying drawings.

### First Embodiment

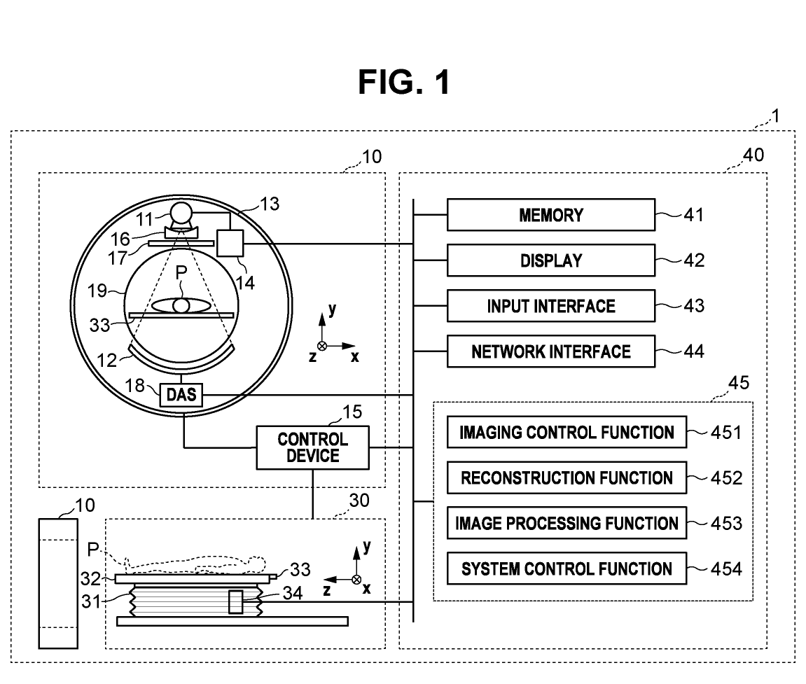

FIG. 1 is a block diagram illustrating a configuration example of an X-ray computed tomography (CT) apparatus 1 according to a first embodiment. FIG. 1 is also applied to second, third, and fourth embodiments.

As illustrated in FIG. 1, the X-ray CT apparatus includes a gantry 10, a couch 30, and a console 40. Although FIG. 1 illustrates a plurality of gantries 10 for convenience of explanation, in practice, there may be one or more gantries 10. The X-ray CT apparatus 1 is an example of a medical image diagnostic apparatus. The X-ray CT apparatus 1 may be not only an X-ray CT apparatus used alone, but also an X-ray CT apparatus used in combination with a positron emission tomography (PET) examination apparatus, or an X-ray CT apparatus used in combination with an angiography apparatus.

The gantry 10 is a scanning device having a configuration for performing X-ray CT imaging of a subject P. The couch 30 is a transport device on which the subject P, who is to be subjected to X-ray CT imaging, is to be placed, and is used for positioning. The console 40 is a computer that controls the gantry 10. For example, the gantry 10 and the couch 30 are installed in a CT examination room, and the console 40 is installed in a control room adjacent to the CT examination room. The gantry 10, the couch 30, and the console 40 are connected to each other by wire or wirelessly so as to be communicable with each other. The console 40 is not necessarily required to be installed in the control room. For example, the console 40 may be installed in the same room as the gantry 10 and the couch 30. Alternatively, the console 40 may be incorporated into the gantry 10.

As illustrated in FIG. 1, the gantry 10 includes an X-ray tube 11, an X-ray detector 12, a rotating frame 13, an X-ray high-voltage device 14, a control device 15, a wedge 16, a collimator 17, and data acquisition circuitry (data acquisition system (DAS)) 18.

The X-ray tube 11 irradiates the subject P with X-rays. Specifically, the X-ray tube 11 includes a cathode that generates thermoelectrons, an anode that receives thermoelectrons emitted from the cathode and generates X-rays, and a vacuum tube that holds the cathode and the anode. The X-ray tube 11 is connected to the X-ray high-voltage device 14 via a high-voltage cable. The X-ray high-voltage device 14 applies a tube voltage between the cathode and the anode. The application of the tube voltage causes thermoelectrons to be emitted from the cathode to the anode. When thermoelectrons are emitted from the cathode and move toward the anode, a tube current flows. When the thermoelectrons collide with the anode, X-rays are generated.

The X-ray detector 12 detects X-rays that have been emitted from the X-ray tube 11 and have passed through the subject P, and outputs an electrical signal corresponding to the dose of the detected X-ray to the data acquisition circuitry 18. The X-ray detector 12 has a structure in which a plurality of X‑ray detection element rows, each arranged in a channel direction, is further arranged in a slice direction (row direction). The X-ray detector 12 is an indirect conversion-type detector including a collimator, a scintillator array, and a photosensor array, for example.

The collimator is disposed on the X-ray incident side of the scintillator array and includes an X-ray shielding plate that absorbs scattered X-rays. There are various types of collimators, such as a one-dimensional collimator, a two-dimensional collimator, and other types of collimators. The scintillator array has a plurality of scintillators. Each scintillator emits light in an amount corresponding to the dose of incident X-rays. The photosensor array converts the light from the scintillators into an electrical signal according to the amount of light. For example, a photodiode may be used as the photosensor. The X-ray detector 12 may also be a direct conversion-type detector.

The rotating frame 13 is an annular frame that supports the X-ray tube 11 and the X-ray detector 12 so as to be rotatable about a rotation axis (Z-axis). Specifically, the rotating frame 13 supports the X-ray tube 11 and the X-ray detector 12 so as to face each other. The rotating frame 13 is supported on a fixed frame (not illustrated) so as to be rotatable around the rotation axis. The control device 15 rotates the rotating frame 13 about the rotation axis, thereby rotating the X-ray tube 11 and the X-ray detector 12 about the rotation axis. The rotating frame 13 is rotated at a constant angular velocity by receiving driving force from a drive mechanism of the control device 15. An opening 19 of the rotating frame 13 defines an image field of view (FOV).

As illustrated in FIG. 1, in the present embodiment, the longitudinal direction of the rotation axis of the rotating frame 13 in a non-tilted state or the longitudinal direction of the couchtop 33 of the couch 30 will be defined as a Z-axis direction, the axis direction perpendicular to the Z-axis direction and horizontal to the floor surface will be defined as an X-axis direction, and the axis direction perpendicular to the Z-axis direction and perpendicular to the floor surface will be defined as Y-axis direction.

The X-ray high-voltage device 14 includes a high-voltage generation device and an X-ray control device. The high-voltage generation device includes electric circuitry including a transformer, rectifier circuitry, and the like, and generates a high voltage to be applied to the X-ray tube 11 and a filament current to be supplied to the X-ray tube 11. The X-ray control device controls the output voltage according to the X-rays emitted by the X-ray tube 11. The high-voltage generation device may be of a transformer type or an inverter type. The X-ray high-voltage device 14 may be provided in the rotating frame 13 of the gantry 10, or in a fixed frame (not illustrated) of the gantry 10.

The wedge 16 adjusts the dose of X-rays with which the subject P is irradiated. Specifically, the wedge 16 attenuates the X-rays so that the dose of X-rays with which the X-ray tube 11 irradiates the subject P has a predetermined distribution. For example, the wedge 16 may be a metal plate such as an aluminum plate, and may take the form of a wedge filter or a bow-tie filter.

The collimator 17 limits the irradiation range of X-rays having passed through the wedge 16. The collimator 17 slidably supports a plurality of lead plates that blocks X-rays, and adjusts the shape of slits formed by the plurality of lead plates. The collimator 17 may also be called an X-ray diaphragm.

The data acquisition circuitry 18 reads, from the X-ray detector 12, an electrical signal corresponding to the dose of the X-rays detected by the X-ray detector 12. The data acquisition circuitry 18 amplifies the read electrical signal and integrates the electrical signal over a view period to collect detection data having a digital value corresponding to the dose of the X-rays over the view period. The detection data is called projection data. The data acquisition circuitry 18 is implemented, for example, by an Application-Specific Integrated Circuit (ASIC) equipped with circuit elements capable of generating projection data. The projection data is transmitted to the console 40 via a non-contact data transmission device or the like.

The control device 15 controls the X-ray high-voltage device 14 and the data acquisition circuitry 18 to perform X-ray CT imaging in accordance with an imaging control function 451 of processing circuitry 45 of the console 40. The control device 15 includes processing circuitry including a central processing unit (CPU), a micro processing unit (MPU), or the like, and driving mechanisms, such as a motor and an actuator. The processing circuitry 45 includes, as hardware resources, a processor, such as a CPU, and memories, such as a read only memory (ROM) and a random access memory (RAM).

The control device 15 executes various functions using a processor that executes programs loaded in the memory. The various functions are not limited to being implemented by a single piece of processing circuitry. The processing circuit may alternatively be configured by combining a plurality of independent processors, each executing a program to implement the respective functions. The control device 15 may also be implemented by an ASIC or a field programmable gate array (FPGA). The control device 15 may also be implemented by another complex programmable logic device (CPLD) or simple programmable logic device (SPLD).

The control device 15 has a function of receiving input signals from an input interface 43 (described below) attached to the console 40 or the gantry 10 and controlling the operation of the gantry 10 and the couch 30. For example, the control device 15 receives an input signal and performs the control of rotating the rotating frame 13, the control of tilting the gantry 10, and the control of operating the couch 30 and the couchtop 33. The control of tilting the gantry 10 is implemented by the control device 15 rotating the rotating frame 13 about an axis parallel to the X-axis direction, based on inclination angle (tilt angle) information input via an input interface attached to the gantry 10. The control device 15 may be provided in the gantry 10 or in the console 40.

The couch 30 includes a base 31, a support frame 32, the couchtop 33, and a couch driving device 34. The base 31 is disposed on the floor surface. The base 31 is a housing that supports the support frame 32 so as to be movable in a vertical direction (Y-axis direction) with respect to the floor surface. The support frame 32 is a frame provided on the top of the base 31. The support frame 32 supports the couchtop 33 so as to be slidable in the rotation axis (Z-axis). The couchtop 33 is a flexible plate on which the subject P is to be placed.

The couch driving device 34 is housed in the housing of the couch 30. The couch driving device 34 is a motor or actuator that generates a driving power to move the support frame 32 and the couchtop 33 on which the subject P is placed. The couch driving device 34 operates under the control of the console 40 or the like.

The console 40 includes a memory 41, a display 42, the input interface 43, a network interface 44, and the processing circuitry 45. Data communication among the memory 41, the display 42, the input interface 43, the network interface 44, and the processing circuitry 45 is performed via a bus (BUS). Although the console 40 is described as being separate from the gantry 10, the gantry 10 may include the console 40 or some of the components of the console 40.

The memory 41 is a storage device, such as a hard disk drive (HDD), a solid state drive (SSD), or an integrated circuitry storage device, that stores various types of information. In addition to HDDs and SSDs, the memory 41 may be a portable storage medium, such as a compact disc (CD), a digital versatile disc (DVD), a Blu-ray (registered trademark) disc (BD), or a flash memory. The memory 41 may also be a drive device that reads and writes various types of information to and from semiconductor memory elements, such as a flash memory or a RAM. The storage area of the memory 41 may be located in the X-ray CT apparatus 1 or in an external storage device connected via a network. The memory 41 stores projection data and reconstructed image data, for example.

The display 42 displays various types of information. For example, the display 42 outputs CT images generated by the processing circuitry 45, a graphical user interface (GUI) for accepting various operations from an operator, and the like. Any type of display may be appropriately used as the display 42. For example, the display 42 can be a liquid crystal display (LCD), a cathode ray tube (CRT) display, an organic electroluminescence display (OELD), or a plasma display.

The display 42 may be installed anywhere in the control room. Alternatively, the display 42 may be installed in the gantry 10. The display 42 may be a desktop type, or may be configured as a tablet terminal or the like capable of wireless communication with the main body of the console 40. Alternatively, the display 42 may be implemented using one or more projectors.

The input interface 43 accepts various input operations from the operator, converts the accepted input operations into electrical signals, and outputs the electrical signals to the processing circuitry 45. For example, the input interface 43 accepts, from the operator, collection conditions for collecting projection data, reconstruction conditions for reconstructing CT images, image processing conditions for generating post-processed images from CT images, and the like. Examples of the input interface 43 that can be used as appropriate include a mouse, keyboard, trackball, switch, button, joystick, touchpad, and touch panel display. In the present embodiment, the input interface 43 is not limited to those equipped with physical operation components, such as a mouse, keyboard, trackball, switch, button, joystick, touchpad, and touch panel display. Examples of the input interface 43 also include electrical signal processing circuitry that receives electrical signals corresponding to an input operation from an external input device provided separately from the console 40 and outputs the electrical signals to the processing circuitry 45. The input interface 43 may be provided in the gantry 10. The input interface 43 may be configured as a tablet terminal or the like capable of wireless communication with the console 40.

The network interface 44 includes a connector conforming to a parallel connection specification or a serial connection specification. If the X-ray CT apparatus 1 is provided in a medical imaging system, the network interface 44 transmits and receives information to and from an external apparatus on the network. For example, under the control of the processing circuitry 45, the network interface 44 receives an examination order for a CT examination from an external apparatus, and transmits detection data acquired by the X-ray CT apparatus 1 and generated raw data or CT image data to the external apparatus.

The processing circuitry 45 controls the overall operation of the X-ray CT apparatus 1 in response to electrical signals of input operations output from the input interface 43. The processing circuitry 45 generates image data based on electrical signals output from the X-ray detector 12. For example, the processing circuitry 45 includes, as hardware resources, processors such as a CPU, MPU, or GPU, and memories such as a ROM or RAM. The processor executes programs loaded in the memory. The processing circuitry 45 includes an imaging control function 451, a reconstruction function 452, an image processing function 453, a system control function 454, and the like.

Each of the functions 451 to 454 is not necessarily implemented by a single piece of processing circuitry. Each of the functions 451 to 454 may be implemented by combining a plurality of independent processors to form processing circuitry, and executing programs by each processor.

The imaging control function 451 includes a function of executing X-ray CT imaging while controlling the X-ray high-voltage device 14, the control device 15, and the data acquisition circuitry 18 in accordance with preset imaging conditions.

The reconstruction function 452 includes a function of performing preprocessing such as logarithmic conversion, offset correction, inter-channel sensitivity correction, beam hardening correction, and the like on the projection data output from the data acquisition circuitry 18. The reconstruction function 452 performs reconstruction on the preprocessed projection data using methods such as filtered back projection method, iterative reconstruction, or machine learning, and generates CT images.

The image processing function 453 includes a function of converting CT images generated by the reconstruction function 452 into cross-sectional image of any cross sections or rendering images from any viewpoints. The conversion is performed based on an input operation received from the operator via the input interface 43. For example, the image processing function 453 applies three-dimensional image processing, such as volume rendering, surface volume rendering, image value projection, multi-planar reconstruction (MPR), and curved MPR (CPR) to the CT image data to generate rendering images from any viewpoints.

The system control function 454 will be described below.

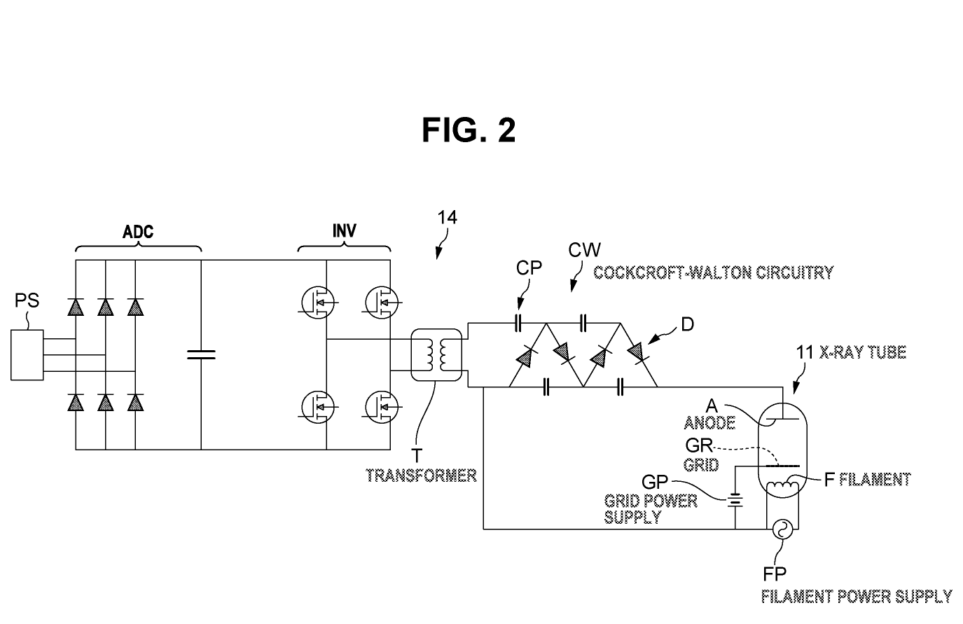

FIG. 2 is a diagram illustrating a circuitry configuration of the X-ray high-voltage device 14 and the X-ray tube 11 according to the first embodiment. The X-ray high-voltage device 14 may be provided not only in an X-ray CT apparatus but also in an X-ray diagnostic apparatus or an angiography apparatus. FIG. 2 is also applied to the second and third embodiments.

As illustrated in FIG. 2, the X-ray high-voltage device 14 includes an alternating-current (AC) power supply PS, AC/direct-current (DC) conversion circuitry ADC, an inverter INV, a transformer T, and Cockcroft-Walton circuitry CW.

The AC power supply PS is a three-phase AC power supply or a single-phase AC power supply. The AC/DC conversion circuitry ADC converts the AC current supplied from the AC power supply PS into DC current. The AC/DC conversion circuitry ADC functions as a DC power supply to be input to the inverter INV.

The inverter INV is full-bridge circuitry that functions as inverter circuitry of the X-ray high-voltage device 14. The inverter INV converts the DC voltage supplied from the AC/DC conversion circuitry ADC into a high-frequency AC voltage and outputs the voltage as an AC pulse power supply. The inverter INV does not have to be full-bridge circuitry, and may be half-bridge circuitry or push-pull circuitry, for example.

The transformer T steps up the AC voltage received from the inverter INV and supplies the stepped-up tube voltage to the X-ray tube 11 via the Cockcroft-Walton circuitry CW. The Cockcroft-Walton circuitry CW converts the AC voltage output from the transformer T into a high-voltage DC voltage and outputs it to the X-ray tube 11 as a tube voltage. The Cockcroft-Walton circuitry CW is voltage-doubling rectifier circuitry that combines a capacitor CP and a diode D. The types of elements, the number of stages, and whether full-wave or half-wave rectification is used are not limited with respect to the Cockcroft-Walton circuitry CW. The Cockcroft-Walton circuitry CW may also have a configuration with two systems (in parallel).

As illustrated in FIG. 2, the X-ray tube 11 includes an anode A, a filament F, and a grid GR disposed between the anode A and the filament F. The anode A is an example of an anode. The grid GR is an example of a grid electrode. The X-ray high-voltage device 14 applies a tube voltage between the anode A and the filament F. The X-ray high-voltage device 14 is an example of a high-voltage generation unit. The tube current is a current that flows between the anode A and the filament F in the X-ray tube 11.

A filament power supply FP applies a filament voltage between both terminals of the filament F. A grid power supply GP applies a grid voltage between the filament F and the grid GR.

The system control function 454 includes a function of enabling the output voltage of the inverter INV at the start of X-ray imaging, and enabling a grid voltage Vg during the rise period of the tube voltage to inhibit the tube current. The system control function 454 also includes a function of disabling the output voltage of the inverter INV when X-ray imaging is ended and discharging electric charge remaining in the high-voltage generation device of the X-ray high-voltage device 14. Details of this function will be described below.

The system control function 454 may be executed by the processing circuitry 45 of the console 40, or may be executed by the control board (microcomputer of the high-voltage generation device) of the X-ray high-voltage device 14, or may be executed by the control device 15. Alternatively, the system control function 454 may be executed by other control circuitry for controlling the X-ray tube 11.

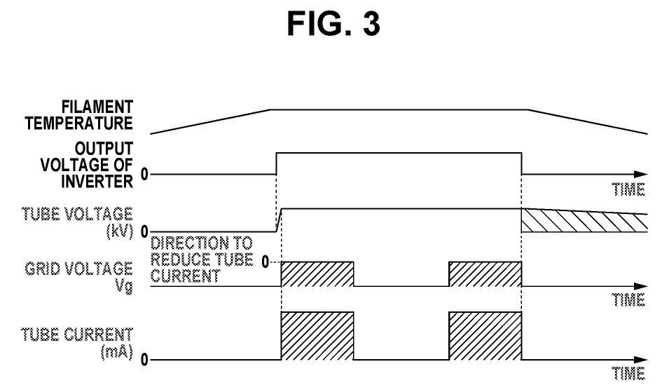

FIG. 3 is a diagram illustrating temporal changes in the output voltage of the inverter INV and the tube current according to the first embodiment. The X-ray CT examination according to FIG. 3 is intended for an operation in which an imaging scan involving a rotation around the torso or head of the subject P is repeated several times, or an operation in which a subject (arteries or veins) into which a contrast agent has been injected is imaged several times at intervals using a wide scan. The X-ray CT examination may be not only continuous X-ray imaging but also pulsed X-ray imaging, and is an example of X-ray imaging.

As illustrated in FIG. 3, when the system control function 454 enables the output voltage of the inverter INV, the tube voltage rises. The output voltage of the inverter INV and an ON state of the tube voltage are maintained until the end of X-ray imaging. During the rise period of the tube voltage, the system control function 454 applies the grid voltage Vg via the grid power supply GP. At this time, the grid voltage Vg is applied to the grid GR as a negative voltage with respect to the filament F. In this way, applying the grid voltage Vg in the direction opposite to the tube voltage makes it possible to inhibit the tube current. This enables the control of on/off of X-ray irradiation.

The on/off operation of the grid power supply GP is faster than the on/off operation of the output voltage of the inverter INV and is thus easier to control, so that X-ray radiation that does not contribute to images during the rise period of the tube voltage can be reduced. In addition, turning on/off the grid power supply GP after the capacitor CP of the Cockcroft-Walton circuitry CW has been charged provides the advantageous effect of accelerating the rise of the tube current in the X-ray tube 11.

On the other hand, at the end of X-ray imaging, the system control function 454 disables the output voltage of the inverter INV while enabling the grid voltage Vg. In other words, this inhibits the tube current within a short time.

Accordingly, the grid voltage Vg is applied until discharge due to leakage current is completed, thus inhibiting X-ray exposure. However, as illustrated on the right side of the tube voltage in FIG. 3, electric charge remains in the capacitor CP of the Cockcroft-Walton circuitry CW, which is high-voltage circuitry (voltage-doubling rectifier circuitry), and discharging the electric charge takes time. During this period, a high voltage is applied to the capacitor CP, the X-ray tube 11, and cables, which may lead to heat generation, failure, and ultimately shortened service life.

The X-ray imaging may be performed intermittently. In that case, in a case where X-ray imaging is temporarily stopped, the system control function 454 keeps the output voltage of the inverter INV enabled and enables the grid voltage Vg. When X-ray imaging is resumed, the system control function 454 disables the grid voltage Vg. In other words, in a case where a plurality of X-ray imaging operations is performed consecutively, the system control function 454 keeps the output voltage of the inverter INV enabled, enables the grid voltage Vg when one X-ray imaging operation is ended, and disables the grid voltage Vg when the next X-ray imaging operation is started.

### Second Embodiment

A second embodiment relates to control for improving the first embodiment. Specifically, when X-ray imaging is ended, a system control function 454 disables the output voltage of an inverter INV while keeping a grid voltage Vg disabled, thus discharging the electric charge remaining in an X-ray high-voltage device 14.

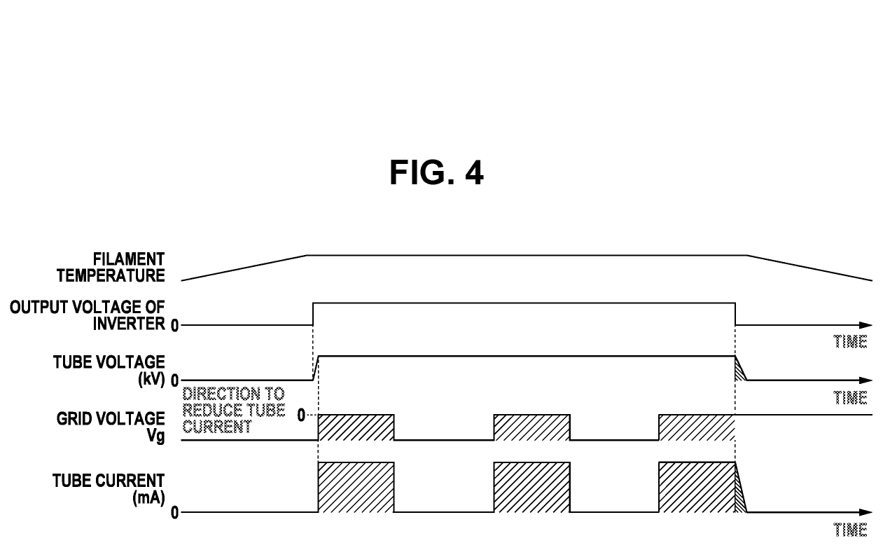

FIG. 4 is a diagram illustrating temporal changes in the output voltage of the inverter INV and the tube current according to the second embodiment. The difference from the first embodiment in FIG. 3 is that the grid voltage Vg is not applied when the final X-ray imaging is ended. In other words, at the end of a series of scans or in a case where an interruption operation is performed during a scan, the output voltage of the inverter INV is disabled to terminate the X-ray irradiation without using the grid voltage Vg to inhibit the tube current.

At this time, the electric charge remaining in a capacitor CP of a Cockcroft-Walton circuitry CW is discharged. This can shorten the rise time and fall time of X-rays and reduce radiation exposure as much as possible without requiring any additional mechanical mechanism. However, as illustrated in FIG. 4, at the end of X-ray imaging, there are fall times for the tube voltage and the tube current, so a subject P may be exposed to some radiation.

### Third Embodiment

A third embodiment relates to control for improving the second embodiment. This is an example in which X-ray irradiation is performed to discharge the electric charge in a Cockcroft-Walton circuitry CW with a subject P moved to a region free from X-ray irradiation or shielded from the X-rays. Specifically at the end of X-ray imaging, a system control function 454 enables a grid voltage Vg, and after a predetermined time, disables the grid voltage Vg and also disables the output voltage of an inverter INV. At the end of X-ray imaging, the system control function 454 may disable the output voltage of the inverter INV during a predetermined time period between enabling and disabling the grid voltage Vg.

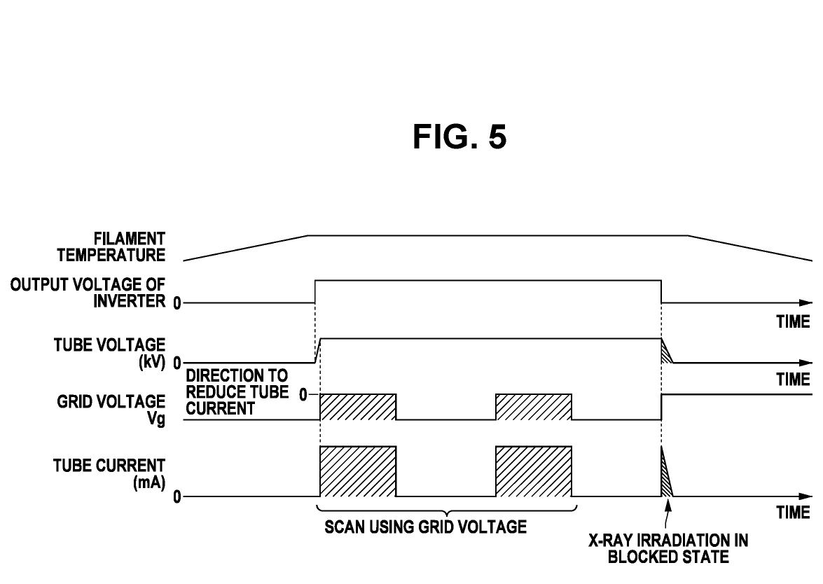

FIG. 5 is a diagram illustrating temporal changes in the output voltage of the inverter INV and the tube current according to the third embodiment. The difference from FIG. 4 corresponding to the second embodiment is that the grid voltage Vg is applied once at the end of the final X-ray imaging, and then is disabled after a predetermined time has lapsed. After a scan is performed with the grid voltage Vg disabled, the grid voltage Vg is enabled to inhibit X-ray irradiation. Then, the grid voltage Vg is disabled and X-ray irradiation is performed with a subject P moved to a region free from X-ray irradiation (moved to a location where X-rays do not reach or removed from the couch 30, or the like).

This configuration allows the subject P within a predetermined time, so that unnecessary exposure to radiation is prevented. In addition, if an interruption operation is performed between scans, electric charge can be released.

An X-ray CT apparatus 1 may further include a display or a speaker that outputs a message prompting the moving of the subject P to a region free from X-ray irradiation. In this case, the system control function 454 causes the display or the speaker to output the message when X-ray imaging is ended. The display and the speaker are examples of an output unit. After receiving the message and detecting that the subject P has been moved to a region free from X-ray irradiation, the system control function 454 disables the grid voltage Vg and disables the output voltage of the inverter INV. The output voltage of the inverter INV may be disabled immediately after outputting the message.

Accordingly, after the subject P has been moved to a region free from X-ray irradiation within a predetermined time, X-ray irradiation can be performed to discharge the electric charge in the Cockcroft-Walton circuitry CW. This prevents unnecessary exposure of the subject P.

The X-ray CT apparatus 1 may further include a member that blocks the X-rays emitted from an X-ray tube 11. In this case, the system control function 454 moves the member to a position where the member can block the X-rays before a predetermined time elapses after X-ray imaging ends.

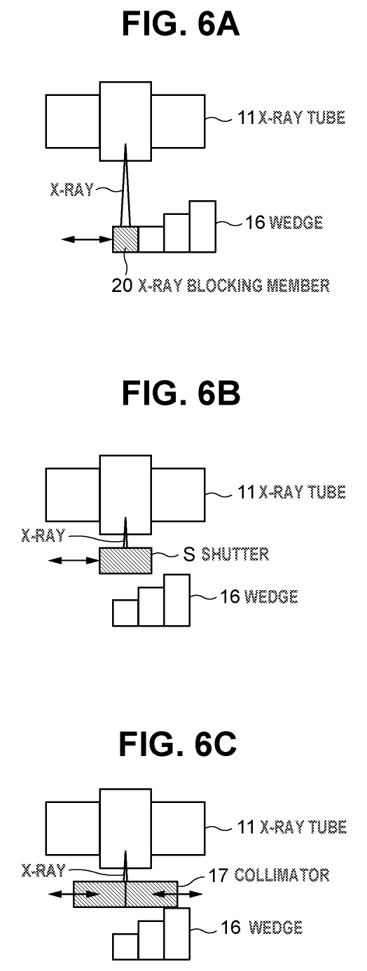

FIGS. 6A to 6C are diagrams illustrating the positional relationship between the X-ray tube 11 and each member according to the third embodiment. As illustrated in FIGS. 6A to 6C, X-rays may be blocked using an X-ray blocking member 20, a shutter S, or a collimator 17. The X-ray blocking member 20, the shutter S, and the collimator 17 are examples of the above-described member.

Referring to FIG. 6A, the X-ray blocking member 20 is provided at a leading end of the wedge 16. Movement of the wedge 16 positions the X-ray blocking member 20 at a location where X-ray blocking member 20 can block X-rays. Referring to FIG. 6B, the shutter S is disposed between the X-ray tube 11 and the wedge 16. The shutter S is moved so that the shutter is arranged at a position where the shutter S can block X-rays. Referring to FIG. 6C, the collimator 17 is disposed between the X-ray tube 11 and the wedge 16. Closing the collimator 17 causes it to be positioned at a location where it can block X-rays.

This makes it possible to discharge the charge from the Cockcroft-Walton circuitry CW, which is high-voltage circuitry, without exposing the subject P to unnecessary radiation and without requiring the subject P to move away to a region free from X-ray irradiation, regardless of the timing of the interruption of X-ray imaging.

### Fourth Embodiment

A fourth embodiment relates to a configuration for improving the first embodiment, by using discharge circuitry EDC to discharge residual electric charge in an X-ray high-voltage device 14a.

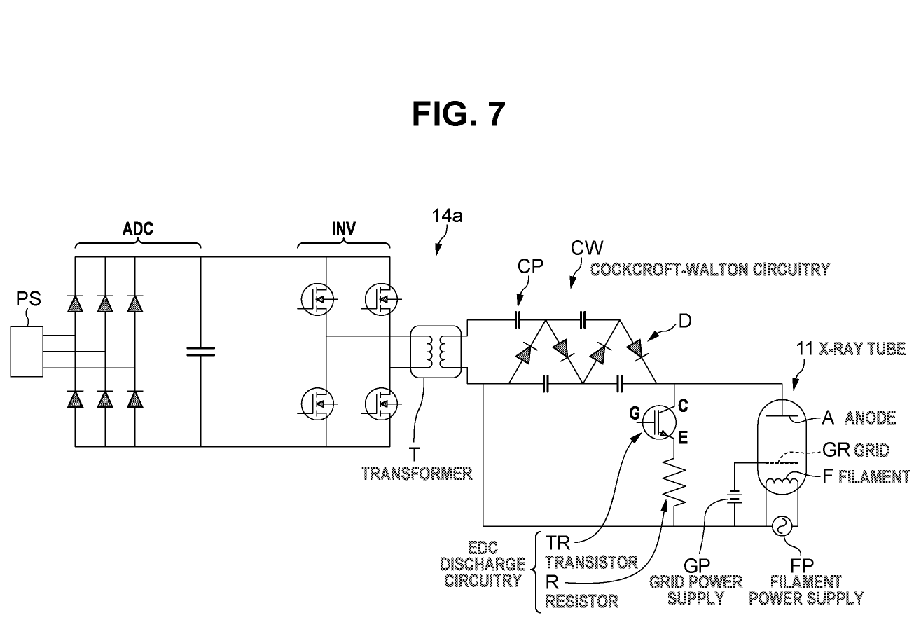

FIG. 7 is a diagram illustrating a circuitry configuration of the X-ray high-voltage device 14a and an X-ray tube 11 according to the fourth embodiment. The difference from the X-ray high-voltage device 14 in FIG. 2 is that the discharge circuitry EDC is connected in parallel with the X-ray tube 11. When X-ray imaging is ended, the discharge circuitry EDC discharges a capacitor CP of Cockcroft-Walton circuitry CW with a grid voltage Vg in an ON state.

The discharge circuitry EDC is configured by connecting a transistor TR and a resistor R in series. The transistor TR is an insulated gate bipolar transistor (IGBT), which enables high-power and high-speed switching. When the output voltage of an inverter INV is disabled with the grid voltage Vg enabled, a system control function 454 applies a positive voltage to a gate G of the transistor TR. At this time, in addition to enabling the grid voltage Vg, the output voltage of a filament power supply FP may be disabled to inhibit X-ray irradiation.

When the gate G is turned on at the transistor TR, a collector current can flow from a collector C to an emitter E. Accordingly, the electric charge remaining in the capacitor CP of the Cockcroft-Walton circuitry CW does not flow to the X-ray tube 11 but flows into the resistor R through the transistor TR. The resistor R consumes the flowed-in electric charge by generating heat or the like. To reduce the amount of heat generated by the resistor R, the resistance value of the resistor R may be large.

This makes it possible to discharge the electric charge from the Cockcroft-Walton circuitry CW without having a mechanical mechanism for moving the wedge 16 or the like to block X-rays. In a case where electric charge is slowly discharged using the discharge circuitry EDC, X-ray irradiation for discharging the electric charge may be performed after safety has been confirmed, such as after the subject P has been moved away to a region free from X-ray irradiation.

According to at least one of the embodiments described above, unnecessary exposure of a subject to radiation in an X-ray CT apparatus can be reduced.

The system control function 454 is an example of a control unit.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray computed tomography apparatus, comprising:
an X-ray tube including an anode, a filament, and a grid electrode, the grid electrode being disposed between the anode and the filament;
a high-voltage generation unit including an inverter and configured to apply a tube voltage between the anode and the filament; and
processing circuitry configured to:
enable an output voltage of the inverter when X-ray imaging is started;
enable a grid voltage during a rise period of the tube voltage to inhibit a tube current when X-ray imaging is started;
disable the output voltage of the inverter when the X-ray imaging is ended; and
discharge electric charge remaining in the high-voltage generation unit when the X-ray imaging is ended.

2. The X-ray computed tomography apparatus according to claim 1, wherein, when the X-ray imaging is ended, the processing circuitry is configured to disable the output voltage of the inverter while keeping the grid voltage disabled to discharge the electric charge.

3. The X-ray computed tomography apparatus according to claim 1, wherein, when the X-ray imaging is ended, the processing circuitry is configured to enable the grid voltage and disables the output voltage of the inverter.

4. The X-ray computed tomography apparatus according to claim 1, wherein, when the X-ray imaging is ended, the processing circuitry is configured to:
enable the grid voltage;
disable the grid voltage after a predetermined time; and
disable the output voltage of the inverter during the predetermined time.

5. The X-ray computed tomography apparatus according to claim 4, further comprising a member configured to block an X-ray emitted from the X-ray tube,
wherein the processing circuitry is configured to move the member to a position where the member is capable of blocking the X-ray, before a lapse of the predetermined time.

6. The X-ray computed tomography apparatus according to claim 4, further comprising an output unit configured to output a message prompting moving of a subject to a region free from X-ray irradiation,
wherein the processing circuitry is configured to cause the output unit to output the message when the X-ray imaging is ended.

7. The X-ray computed tomography apparatus according to claim 6, wherein, after detection of the subject having been moved to the region free from X-ray irradiation, the processing circuitry is configured to:
disable the grid voltage; and
disable the output voltage of the inverter.

8. The X-ray computed tomography apparatus according to claim 3,
wherein the high-voltage generation unit includes rectifier circuitry including a capacitor, and
wherein the X-ray computed tomography apparatus further comprises discharge circuitry configured to discharge the capacitor with the grid voltage in an ON state when the X-ray imaging is ended.

9. The X-ray computed tomography apparatus according to claim 1, wherein the processing circuitry is configured to:
keep the output voltage of the inverter enabled in a case where the X-ray imaging is temporarily stopped;
enable the grid voltage when the X-ray imaging is stopped; and
disable the grid voltage when the X-ray imaging is resumed.

10. The X-ray computed tomography apparatus according to claim 1, wherein the processing circuitry is configured to:
keep the output voltage of the inverter enabled in a case where a plurality of X-ray imaging operations is performed consecutively;
enable the grid voltage when one X-ray imaging operation is ended; and
disable the grid voltage when a next X-ray imaging operation is started.

11. A method for controlling a high-voltage generation device configured to supply power to an X-ray tube including a filament, an anode, and a grid electrode, the grid electrode being disposed between the filament and the anode, the high-voltage generation device including an inverter and configured to apply a tube voltage between the filament and the anode, the method comprising:
enabling an output voltage of the inverter when X-ray imaging is started and enabling a grid voltage during a rise period of the tube voltage to inhibit a tube current; and
disabling, when the X-ray imaging is ended, the output voltage of the inverter and discharging electric charge remaining in the high-voltage generation device.

12. A non-transitory computer-readable storage medium storing a program that causes a computer to execute a method for controlling a high-voltage generation device configured to supply power to an X-ray tube including a filament, an anode, and a grid electrode, the grid electrode being disposed between the filament and the anode, the high-voltage generation device including an inverter and configured to apply a tube voltage between the filament and the anode, the method comprising:
enabling an output voltage of the inverter when X-ray imaging is started and enabling a grid voltage during a rise period of the tube voltage to inhibit a tube current; and
disabling, when the X-ray imaging is ended, the output voltage of the inverter and discharging electric charge remaining in the high-voltage generation device.
